(19) United States
(12) Patent Application Publication
(10) Pub. No.: US 2022/0305150 A1
(43) Pub. Date: Sep. 29, 2022

(54) NOVEL TIN-117M COLLOID FORMULATION WITH THE ABILITY TO DISTINGUISH IT FROM EXISTING TIN-117M COLLOID FORMULATIONS

(71) Applicant: SERENE, LLC, The Woodlands, TX (US)

(72) Inventors: Nigel Raymond Stevenson, Sugar Hill, GA (US); Gilbert R. Gonzales, Tucson, AZ (US); Jaime Simón, Angleton, TX (US)

(21) Appl. No.: 17/619,921

(22) PCT Filed: Jun. 18, 2020
(86) PCT No.: PCT/US2020/038369

§ 371 Date: Dec. 16, 2021

Related U.S. Application Data
(60) Provisional application No. 62/862,969, filed on Jun. 18, 2019.

(51) Int. Cl. A61K 51/12; A61K 33/00; A61K 47/12; A61K 9/00

(52) U.S. Cl. CPC A61K 51/1217; A61K 33/00; A61K 47/12; A61K 9/0085

(57) ABSTRACT

A composition comprising including a homogeneous tin-117m colloid comprising tin-117m, and an ascorbic acid is provided, the composition naturally has a white coloration. The composition is visually distinct from a conventional homogeneous tin-117m colloid because conventional colloids have an orange-yellow color. The addition of ascorbic acid changes the color of the composition without adding toxicity and without changing the therapeutic effects of the homogeneous tin-117m colloid. A use and a method of making the same is also provided.

### CROSS REFERENCE TO RELATED APPLICATIONS

This application is an international patent application, and claims the benefit of the filing date of U.S. Provisional Patent Application No. 62/862,969, filed Jun. 18, 2019, the entire contents of which is hereby incorporated by reference.

### BACKGROUND

A Homogeneous Tin-117m Colloid (HTC; commercial name: Synovetin OA™) is used to treat canine osteoarthritis and other veterinary arthritides. Homogeneous Tin-117m Colloid may also be used to treat human patients, but there is a need to distinguish the veterinary and human products. In addition, it is well known that the veterinary HTC will change color over time, and this can give the impression that the material is no longer useable. Accordingly, there is an additional need for a homogeneous tin-117m colloid that has a stable color.

Distinguishing between homogeneous tin-117m colloid to be used in humans and homogeneous tin-117m colloid to be used for veterinary purposes can be done by changing the color of the colloid. The addition of a minute amount of non-toxic and therapeutically non-functional (i.e., inert) ascorbic acid has the effect of changing the color of the HTC solution from yellow-orange to a consistent white. This is due to the mechanism of reduction of the trace amount of free (excipient) iodine to iodide in solution. This new product (AHTC) is used in human clinical trials to treat and image osteoarthritis, rheumatoid arthritis, and other arthritides, and will be used for several oncologic, therapeutic, and imaging application.

The need for a stable white coloration of a tin-117m colloid includes the reduction or prevention of mixing up between the two types of radioactive material, i.e., giving animal tin-117m colloid to humans and vice versa. Also, as stated above, it is well known that the veterinary HTC will change color over time, and this can give the impression that the material is no longer useable.

Additionally, once the color of the HTC solution has been changed from yellow-orange to a consistent white (due to the addition of ascorbic acid as described above), the solution is then able to be changed to any other color that may be desired. As a result, this may allow for as many differently-colored formulations as may be desired.

### SUMMARY OF THE INVENTION

In one embodiment, a composition including ascorbic acid added to a homogeneous tin-117m colloid is provided. The homogeneous tin-117m colloid includes tin-117m. The composition may be used for imaging and treatment of osteoarthritis, rheumatoid arthritis, and other arthritides. The composition includes a consistent white color that distinguishes the composition formulation from tin-117m colloids without ascorbic acid.

In a related embodiment, the composition includes a white coloration, and further includes at least 0.34 milligrams of ascorbic acid per milliliter of composition. In addition, the composition has a consistent colorimetric value.

In a related embodiment, the composition is near or exactly homogeneous and the tin-117m colloid has a dispersed phase colloid size of between 2 μm and 20 μm, inclusively.

In a related embodiment, the composition is near or exactly homogeneous and the tin-117m colloid has a dispersed phase colloid size of between 2 μm and 50 μm, inclusively.

In a related embodiment, the composition may be suitable for use in treating inflammatory arthritides, in imaging the spread of cancer cells, or in treating the spread of cancer cells. In some embodiments, the imaging of the spread of cancer cells or the treating of the spread of cancer cells occurs in body cavities and closed body spaces. In some embodiments, the body cavities and closed body spaces are selected from the group consisting of an intrathecal space, potential spaces, and a peritoneal cavity.

In a related embodiment, the white color of the composition is independent from a specific activity of the tin-117m.

In a related embodiment, the specific activity of the tin-117m used to form the homogeneous tin-117m colloid is produced by nuclear reactors, and has a usable range of up to 10 Ci/gm.

In a related embodiment, the specific activity of the tin-117m used to form the homogeneous tin-117m colloid is produced by cyclotrons, and is between 3 Ci/gm and 10 Ci/gm, inclusively.

In a related embodiment, the specific activity of the tin-117m used to form the homogeneous tin-117m colloid is produced by cyclotrons, and is between 10 Ci/gm and 100 Ci/gm, inclusively.

In a related embodiment, the specific activity of the tin-117m used to form the homogeneous tin-117m colloid is produced by cyclotrons, and is between 100 Ci/gm and 21,000 Ci/gm, inclusively.

In a related embodiment, the specific activity of the tin-117m used to form the homogeneous tin-117m colloid is produced by cyclotrons, and is between 100 Ci/gm and 21,000 Ci/gm, inclusively, and the tin-117m is coated on non-radioactive tin-117.

In a related embodiment, the tin-117m is coated on non-radioactive tin-117.

In another embodiment, the use of the composition as a human or veterinary medicine is provided.

In another embodiment, a method of making a composition is provided. The method includes adding ascorbic acid to a tin-117m colloid formulation to form a consistent white color composition of the tin-117m colloid formulation.

In a related embodiment, the method further comprises changing the color of the composition from white to a color other than white by adding a food dye of a desired color.

### BRIEF DESCRIPTION OF THE DRAWINGS

The embodiments may be better understood with reference to the following drawings and description. The components in the figures are not necessarily to scale. Moreover, in the figures, like-referenced numerals designate corresponding parts throughout the different views.

FIG. 1 illustrates an example of a homogeneous tin-117m colloid without ascorbic acid.

FIG. 2 illustrates an example of a homogenous tin-117m colloid with added ascorbic acid.

### DETAILED DESCRIPTION

By way of an introductory example, a homogeneous tin-117m colloid with added ascorbic acid is provided. A non-limiting example of making the homogeneous tin-117m colloid with added ascorbic acid is achieved by the following method.

### Method of Making

Formation of Homogeneous Tin Colloid (HTC):

4 M urea, SnI₄ in 0.05 M HCl and ^{117m}SnI₄ in 1M HCl is added in microwave vial. The microwave vial may have a volume of, in a non-limiting example of 5 mL or 20 mL. The vial is placed in a commercial microwave reactor with stirring, high absorption and FHT=“on” (these settings provide frequency stabilization for water molecular heating). A reaction occurs in this manner at 90° C. for 4 hours. The drug substance and formulated drug product are formed simultaneously during this reaction. Following the reaction, the products of the reaction are sterilized at 135° C. for 30 minutes.

Addition of Ascorbic Acid to Form AHTC (Commercial Name: Synovacoll™):

100 μL (5 mL batch) or 400 μL (20 mL batch) of 0.1 M ascorbic acid solution is aseptically added to the bulk vial, followed by mixing. In this example, the amount of added ascorbic acid to the bulk vial corresponds to 0.34 mg/mL in the final product.

For quality control purposes, samples are aseptically removed from the bulk vial. The bulk product is also assayed for total batch radioactivity, volume, and radioactivity concentration. The assays are tested for particle size, pH, endotoxin, and undergo a visual inspection to confirm that the bulk final product is within specification.

Individual unit doses are aseptically dispensed into pre-labelled open depyrogenated and sterile patient dose final container closures, for example 3 mL serum vials.

The patient dose final container closures are terminally sterilized, using dry heat sterilization.

Of particular note is the change in color from yellow-orange (HTC) to white/cream or pale red (AHTC). All other characteristics were tested and when compared with HTC were found to remain unchanged.

The method may optionally include changing the color of the AHTC composition by adding an additive, such as a food dye to the AHTC composition.

The specifications for the AHTC is shown below in TABLE 1:

TABLE 1

| AHTC Specification # | | |
|---|---|---|
| Test | Method | Specification |
| Appearance | Visual inspection | White turbid particles and white, cream or |
| | | pale- red colored solution |
| pH | microprobe | 6.5-9.0 |
| Median Particle Size | Horiba Model LA-300 | 2.5-6 μm |
| (PS) | Particle Size Analyzer | |
| Particle Size Range | Horiba Model LA-300 | ≥90% above 1.5 μm |
| (D10 to D90) | Particle Size Analyzer | ≥90% below 20 μm |
| Endotoxin | Kinetic-chromogenic | <58 EU/mL |
| | (Charles River Endosafe PTS) | |
| Sterility | ISO 20857 | SAL ≥ 10⁻⁶ |
| | | Sterile based on visual assessment of BI colors |
| Free Sn | Radioactivity measurement | ≤0.2% |

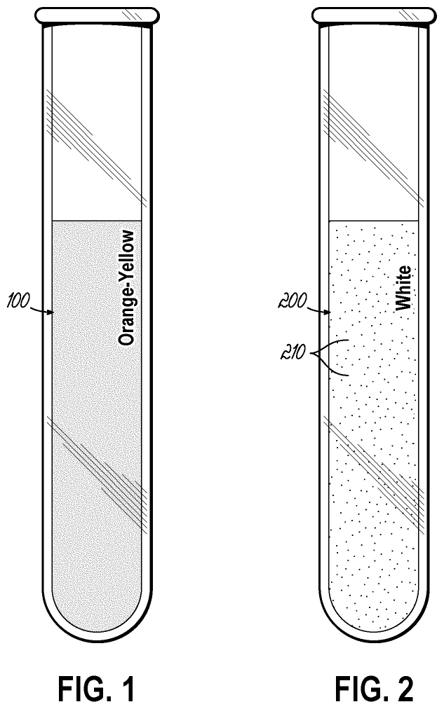

Turning to the figures, FIG. 1 illustrates an example of a homogeneous tin-117m colloid 100 without the addition of ascorbic acid. The homogeneous tin-117m colloid 100 has an orange-yellow color.

FIG. 2 illustrates an example of a homogeneous tin-117m colloid 200 with the addition of ascorbic acid 210. The ascorbic acid 210 turns the color of the composition, that is, the solution of the tin-117m colloid 200 and the ascorbic acid 210, to a creamy white color.

The homogeneous tin-117m colloid 200 and ascorbic acid 210 composition retains its creamy white color for a longer duration than the homogeneous tin-117m colloid without ascorbic acid retains its orange-yellow color. The deterioration of the orange-yellow color of the homogeneous tin-117m colloid 100 may cause physicians using it to consider that it has lost its therapeutic effects. The ascorbic acid 210 is non-toxic and therapeutically inert, and accordingly, the addition of ascorbic acid 210 to the homogeneous tin-117m colloid does not lead to an increased risk of harm to the patient upon which it is used nor does it affect the therapeutic effectiveness of the homogeneous tin-117m colloid 200.

In some examples, the composition is near or exactly homogeneous and the tin-117m colloid has a dispersed phase colloid size of between 2 μm and 20 μm, inclusively. Alternatively or in addition, the composition is near or exactly homogeneous and the tin-117m colloid has a dispersed phase colloid size of between 2 μm and 50 μm, inclusively.

The tin-117m specific activity is important in the therapeutic outcome. Low specific activity (S.A.) in the range of 3 Ci/gm up to 10 Ci/gm are easily achievable using nuclear reactor irradiation of starting material. High specific activity produced tin-117m in the range of 10 Ci/gm to 100 Ci/gm and with much higher S.A. being produced using cyclotrons. Very high S.A. Sn-117m can used for ablation of thickened synovial membranes with severe fibrosis of the synovium in conditions such as chronic pediatric hemophilia synovitis. However, it has been found that the white coloration of the homogeneous tin-117m colloid 200 is unaffected by and is independent of the specific activity of the tin-117m used therein.

In some examples, the tin-117m used in the homogeneous tin-117m colloid 200 is coated on non-radioactive tin-117.

In some examples, non-toxic additives may be added to the composition. For example, food dyes may be added to the white, homogeneous tin-117m colloid 200 with ascorbic acid 210 composition to change the color of the composition to any color desired. The natural, white color of the homogeneous tin-117m colloid 200 with ascorbic acid 210 composition allows for the food dyes, for example, to more easily change the color of the composition rather than the orange-yellow natural coloring of the homogeneous tin-117m colloid 100 without ascorbic acid composition.

The homogeneous tin-117m colloid 200 with ascorbic acid 210 composition may be used as a medicine to treat humans or animals. Particularly, in some examples, the homogeneous tin-117m colloid 200 with ascorbic acid 210 composition may be used to treat humans or animals for osteoarthritis, rheumatoid arthritis, and other arthritides. Alternatively or in addition, the homogeneous tin-117m colloid 200 with ascorbic acid 210 composition may be used for several oncologic, therapeutic, and imaging application. For example, the homogeneous tin-117m colloid 200 with ascorbic acid 210 composition may be used for the imaging of the spread of cancer cells or the treating of the spread of cancer cells occurs in body cavities and closed body spaces. In some embodiments, the body cavities and closed body spaces are selected from the group consisting of an intrathecal space, potential spaces, and a peritoneal cavity.

### In-Vivo Comparison of HTC and AHTC

The homogeneous tin-117m colloid 200 with the addition of ascorbic acid 210 does not affect the actual colloid formation in any way. A rat toxicity study was performed to evaluate an effect of adding the ascorbic acid to HTC. Seven male and seven female Sprague Dawley rats were used in the study. At week 0, the animals received intra-articular (IA) injections of HTC into the left knee and AHTC into the right knee as indicated in Table 1. The dose was the highest expected human patient dose (6 mCi) scaled down for use in a rat weighing approximately 250 grams (nominally 20 μCi). Sacrifice occurred within 2 days of Day 42. At sacrifice, all knees were collected and placed in 10% neutral buffered formalin (NBF). Once the radioactivity decayed to an acceptable level, the knees were shipped for processing. Slides from 28 knee joints (14 left knees and 14 right knees) from 7 male and 7 female Sprague Dawley rats were evaluated for treatment-related effects. Studying the slides produced several observations. No treatment-related cartilage changes were observed in knees injected with Sn-117m colloid or Sn-117m colloid with ascorbic acid. No significant differences were observed from injection of Sn-117m colloid with or without ascorbic acid. Age-related, spontaneous cartilage degeneration (proteoglycan and chondrocyte loss in upper layers) was observed on the medial and lateral tibia in areas not protected by meniscus in 3 right knees and 2 left knees from male rats. Age-related cartilage degenerative changes similar to these have been reported in several rat strains and are commonly seen in time course studies (Smale, 1995). Low levels of synovitis were observed in 2 to 4 joints per treatment in male and female rats; these lesions were considered to be associated with the trauma of injection, and it has been shown that any injection (e.g., saline) into the joint can cause this type of inflammatory response (Emami, 2018). One male rat had mild collagen degeneration and fibroblast proliferation at the cruciate insertions as a result of trauma from injection. Accordingly, the study concluded that knees injected with HTC or AHTC had similar histopathology with no treatment-related cartilage changes and only very minimal to mild synovial inflammation. No statistical difference between the two sets was observed.

### Sn-117m Specific Activity

Research nuclear reactor production of Sn-117m is limited to a maximum of 10 Ci/gm in practice when the time includes to usability of the isotope included post end-of-bombardment, cooling, shipping and preparation for product manufacturing takes into account the Sn-117m half-life of 14 days. An AHTC final product can currently include nuclear reactor Sn-117m and additionally a 10 Ci/gm to 100 Ci/gm nuclear reactor produced Sn-117m is theoretically possible. A high specific activity (HSA) Sn-117m is achievable using cyclotrons that can create a usable AHTC in the range of 10 Ci/gm or less, for example between 3 Ci/gm and 10 Ci/gm, inclusively, as well as between 10 Ci/gm and 100 Ci/gm, inclusively, where the specific activity is not too strong that radiation damage to the synovium might take place from either the gamma photons or conversion electrons that are emitted. HSA cyclotron-produced Sn-117m can be produced at specific activities of up to 21,000 Ci/gm but theoretically a therapeutic AHTC with Sn-117m specific activity between 10 Ci/gm and 100 Ci/gm, inclusively, can be produced for treatment of arthritis or radiation ablation in certain arthritis conditions such as severe pediatric hemophilic arthropathy.

To clarify the use of and to hereby provide notice to the public, the phrases “at least one of <A>, <B>, . . . and <N>” or “at least one of <A>, <B>, . . . <N>, or combinations thereof” or “<A>, <B>, . . . and/or <N>” are defined by the Applicant in the broadest sense, superseding any other implied definitions hereinbefore or hereinafter unless expressly asserted by the Applicant to the contrary, to mean one or more elements selected from the group comprising A, B, . . . and N. In other words, the phrases mean any combination of one or more of the elements A, B, . . . or N including any one element alone or the one element in combination with one or more of the other elements which may also include, in combination, additional elements not listed.

While various embodiments have been described, it will be apparent to those of ordinary skill in the art that many more embodiments and implementations are possible. Accordingly, the embodiments described herein are examples, not the only possible embodiments and implementations.

1. A composition comprising:
a homogeneous tin-117m colloid comprising tin-117m; and
an ascorbic acid,
wherein the composition has a white coloration.

2. The composition in claim 1, wherein the ascorbic acid is present in an amount of at least 0.34 mg of the ascorbic acid per 1 mL of the composition.

3. The composition in claim 1, wherein the tin-117m colloid has a dispersed phase colloid size of between 2 μm and 20 μm, inclusively.

4. The composition in claim 1, wherein the tin-117m colloid has a dispersed phase colloid size of between 2 μm and 50 μm, inclusively.

5. The composition in claim 1, wherein the composition is suitable for use in treating inflammatory arthritides, in imaging the spread of cancer cells, or in treating the spread of cancer cells.

6. The composition of claim 5, wherein the imaging of the spread of cancer cells or the treating of the spread of cancer cells occurs in body cavities or closed body spaces.

7. The composition of claim 6, wherein the body cavities and closed body spaces are selected from the group consisting of an intrathecal space, potential spaces, and a peritoneal cavity.

8. The composition in claim 1, wherein the white coloration is independent from a specific activity of the tin-117m.

9. The composition in claim 1, wherein the specific activity of the tin-117m is produced by nuclear reactors in the usable range of up to 10 Ci/gm.

10. The composition in claim 1, wherein the specific activity of the tin-117m is produced by cyclotrons, and is between 3 Ci/gm and 10 Ci/gm, inclusively.

11. The composition in claim 1, wherein the specific activity of the tin-117m is produced by cyclotrons, and is between 10 Ci/gm and 100 Ci/gm, inclusively.

12. The composition in claim 1, wherein the specific activity of the tin-117m is produced by cyclotrons, and is between 100 Ci/gm and 21,000 Ci/gm, inclusively.

13. The composition in claim 1, wherein the specific activity of the tin-117m is produced by cyclotron, is between 100 Ci/gm and 21,000 Ci/gm, and the tin-117m is coated on non-radioactive tin-117.

14. A use of the composition of claim 1 in human or veterinary medicine.

15. A method comprising:
adding ascorbic acid to a tin-117m colloid formulation to create composition having a consistent white color.

16. The method of claim 15, further comprising subsequently changing the color of the composition to a color other than white by adding a food dye.
